(19) United States
(12) Patent Application Publication
(10) Pub. No.: US 2008/0245135 A1
(43) Pub. Date: Oct. 9, 2008

(54) MICROFLUIDIC ENCAPSULATED NEMS RESONATORS

(75) Inventors: Keith Aubin, Burlington, MA (US); Bojan (Rob) Ilic, Ithaca, NY (US); Seung-Min Park, Ithaca, NY (US); Harold G. Craighead, Ithaca, NY (US)

Correspondence Address: SCHWEGMAN, LUNDBERG & WOESSNER, P.A., P.O. BOX 2938, MINNEAPOLIS, MN, 55402, US

(73) Assignee: Cornell Research Foundation, Inc.

(21) Appl. No.: 11/940,865

(22) Filed: Nov. 15, 2007

Related U.S. Application Data
(60) Provisional application No. 60/859,138, filed on Nov. 15, 2006.

(51) Int. Cl. G01N 29/036; G01N 33/02

(52) U.S. Cl. 73/61.49; 422/68.1

(57) ABSTRACT

A device includes a microfluidic channel and a nanoelectromechanical mass detector encapsulated within the microfluidic channel. Multiple microfluidic channels may be included with multiple nano electromechanical mass detectors encapsulated within each microfluidic channel. A method of detecting masses includes delivering a sample via the microfluidic channel to the nano electromechanical mass detectors and creating a pressure within the microfluidic channel that significantly reduces viscous damping effects on the mass detector. The detector may be actuated and response measured.

### GOVERNMENT FUNDING

The invention described herein was made with Government support by the Defense Advanced Research Projects Agency, through an Office of Naval Research grant number N00014-97-10-0779, and by the National Science Foundation, under contract number ECS-9876771. The United States Government has certain rights in the invention.

### BACKGROUND

Science and industry have developed a need for the ability to accurately detect and measure very small quantities of chemical or biological material. Where it was once adequate to measure quantities in micrograms, today, many applications require the detection of a single cell, subcellular unit, or other small quantity of material, sometimes on the order of 10⁻¹⁵ grams.

For example, within the food industry, even small quantities of particular biological cells or toxins can be harmful or dangerous to mammals. One such well recognized harmful organism is Escherichia coli or E. coli bacteria. Popular media attention concerning the presence of E. coli in meat products, apple juice and alfalfa sprouts has heightened consumer sensitivities.

Sensors for detecting gasses using micromechanical cantilevers detect static beam deflection upon exposure of the beam to gases and vapors and measuring resonance frequency shifts based on changes of beam mass due to absorption. Absorbent beams are exposed to a chemical vapor for a period of time (noted in one instance to be several hours) and measurements are taken before the chemicals have evaporated from the beam. Absorption is recognized as largely a result of Lennard-Jones potential, wherein at close distances, nearby molecules repel and at larger distances, the molecules are attracted to each other. In many cases, absorption of molecules onto a surface can be readily reversed by merely heating the system or exposing the system to a vacuum.

Biochemically induced surface stresses in a cantilever array utilize absolute deflection of a beam as it relates to ligand binding in a liquid environment. Detecting mass differences using static deflection of a beam typically requires a more robust beam. In many cases, this means that the beam is dimensionally rather large or the material of which the beam is fabricated has a relatively high Young's Modulus of elasticity. Large beams or those having high Young's Modulus of elasticity can lack the sensitivity needed to detect small quantities of target substances. In addition, beams that acquire additional mass through the process of absorption often require lengthy exposure time to the target substance to accumulate a detectable amount. For the reasons stated above, and for other reasons stated below which will become apparent to those skilled in the art upon reading and understanding the present specification, there is a need in the art for a highly sensitive detection system and method that permits the rapid detection of biological or chemical material.

### BRIEF DESCRIPTION OF THE DRAWINGS

FIG. 1 is a block schematic diagram of a system that detects analytes in a sample according to an example embodiment.

FIGS. 2A, 2B, 2C, 2D, 2E, 2F, 2G, 2H, and 2I illustrate process steps for making an encapsulated mass sensor in a channel according to an example embodiment.

FIG. 3 is a schematic diagram of an array of encapsulated mass sensors in multiple channels with devices and valves for providing sample fluid, purging gas and a vacuum pump according to an example embodiment.

FIG. 4 is a block perspective view of a cantilever beam illustrating dimensions according to an example embodiment.

FIGS. 5A and 5B illustrate a cantilever beam having a binding partner according to an example embodiment.

### DETAILED DESCRIPTION

In the following description, reference is made to the accompanying drawings that form a part hereof, and in which is shown by way of illustration specific embodiments which may be practiced. These embodiments are described in sufficient detail to enable those skilled in the art to practice the invention, and it is to be understood that other embodiments may be utilized and that structural, logical and other changes may be made without departing from the scope of the present invention. The following description of example embodiments is, therefore, not to be taken in a limited sense, and the scope of the present invention is defined by the appended claims.

Devices and methods are described for detecting an analyte using nano electromechanical system (NEMS) or micro electromechanical system (MEMS) mass sensors. In various embodiments, microfluidic channels are provided with such mass sensors to deliver fluid mixtures of functional and analyte proteins, as well as washing compounds. The microfluidic channels and mass sensors are encapsulated to facilitate the creation of vacuum conditions after the mass sensors have been exposed to a sample to minimize viscous damping of the sensors.

An array of analyte detectors is also disclosed. An array can include a plurality of cantilever beams, a plurality of immobilized binding partners and a sensor responsive to light reflected by a particular beam. Each beam resonates at a particular frequency under ambient conditions. Each beam has an immobilized binding partner on a surface. Each binding partner binds to a predetermined analyte. The sensor generates an output signal based on a resonant frequency of a particular beam.

In some embodiments, a microfluidic channel is formed with a nanoelectromechanical sub-attogram mass detector encapsulated within the microfluidic channel. Multiple microfluidic channels may be included with multiple nanoelectromechanical sub-attogram mass detectors encapsulated within each microfluidic channel. A method of detecting sub-attogram masses includes delivering a sample via the microfluidic channel to the nanoelectromechanical mass detectors and creating a pressure within the microfluidic channel that significantly reduces viscous damping effects on the mass detector. The detector may be actuated and response measured.

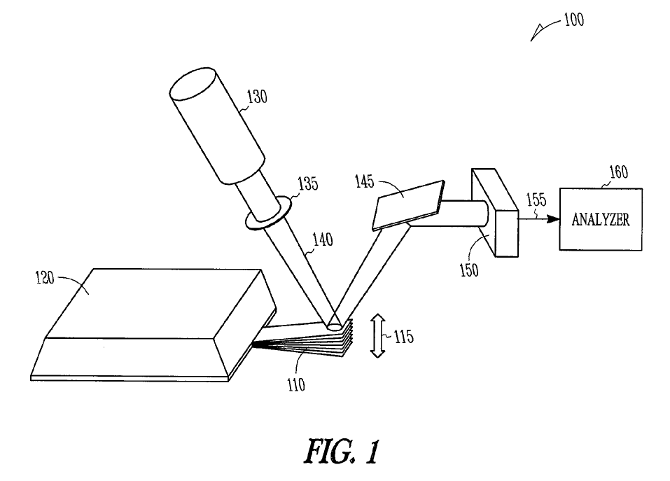

FIG. 1 is a block schematic representation of measurement system 100 for a mass detector. A cantilever beam 110 is affixed on one end to a support such as substrate 120. The other end of beam 110 is free to move in the directions indicated by arrow 115. Beam 110 vibrates at a resonant frequency when driven by ambient environmental conditions or when driven externally, by, for example, a piezoelectric device. In the embodiment shown, a laser 130 projects light 140, optionally through lens 135, onto the free end of beam 110. Lens 135 focuses the light onto the apex of beam 110. Light 140 is reflected by beam 110. A mirror 145 can redirect light 140 to illuminate a sensor 150. Sensor 150, which may be photodiode in one embodiment, generates an output signal 155 based on the vibrations of cantilever beam 110. Spectrum analyzer 160 processes output signal 155 and yields useful information.

The resonant frequency of the beam is a function of, inter alia, the mass on the beam. The photodiode generates an output signal based on light reflected from the apex of the beam. The photodiode output is a function of the frequency of vibration of the beam. At least one surface of the beam includes an immobilized binding partner. The binding partner is selected so as to bind with a particular analyte, or analytes, which in turn, increase the mass of the beam.

In one embodiment, beam 110 is fabricated of silicon nitride, which may be a low stress silicon nitride. Beam 110 may also be fabricated of other materials, including for example, silicon, silicon dioxide, silicon carbide, polysilicon, carbon, diamond like carbon (DLC) film, metal, gallium arsenide or other conductor or semiconductor material. In various embodiments, the material used for beam 110 is conducive to photolithography processes and etching to release beam 110 from the surrounding structure.

Micromachining techniques, or other suitable technology may be used to fabricate beam 110. Beam 110 may be fabricated using either bulk or surface silicon micromachining technology. In the embodiment shown, beam 110 is substantially linear. Alternatively, beam 110 may include a helical section or multiple anchor points with various modes of freedom to enable greater sensitivity. Beam 110 can have different cross sectional shapes, including, for example, a rectangular, square or round cross section.

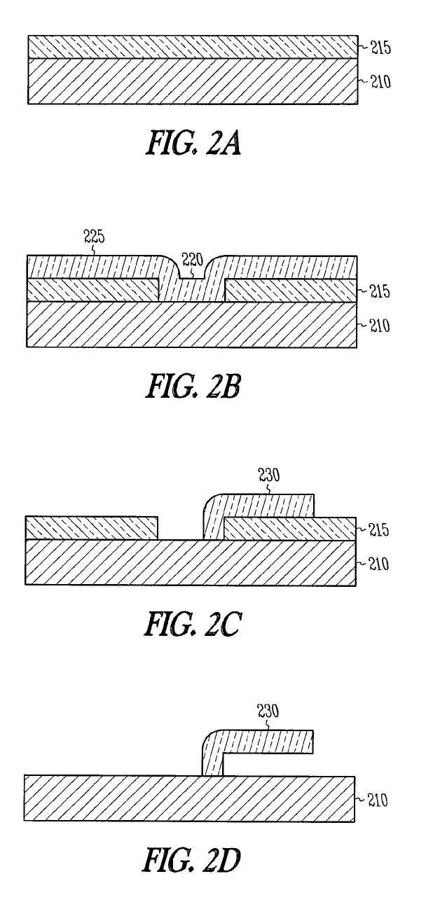

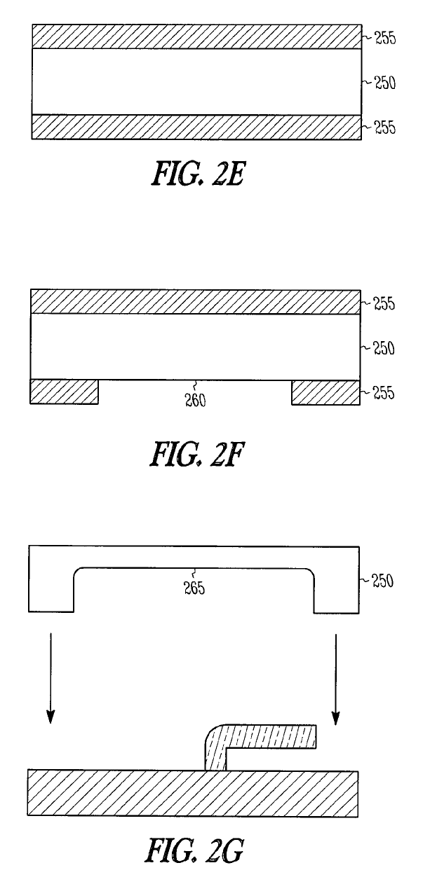

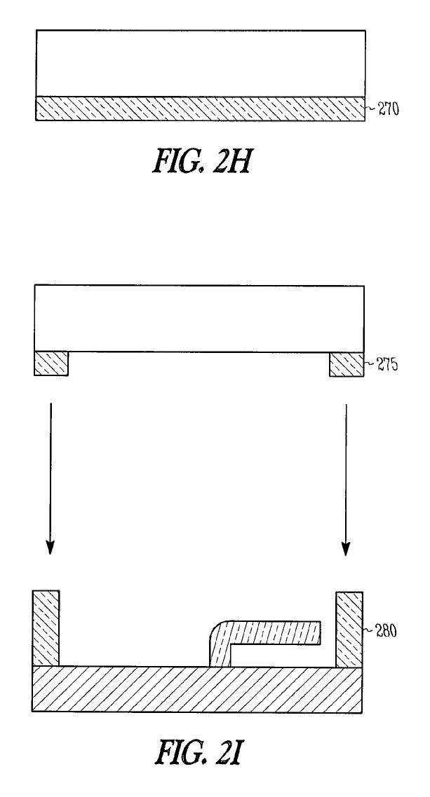

In one embodiment, one or more independently accessible microfluidic channels may be created in at least the following manner. A first wafer may be patterned with one or more channels and mass sensors, and a second wafer may be used to encapsulate the first wafer to provide a sealed environment, enabling exposure of the sensor to an analyte in a sample fluid, removal of the fluid, creation of at least a partial vacuum, and detecting motion of the sensor. Selected stages of the device during the process are illustrated in cross section in FIGS. 2A, 2B, 2C, 2D, 2E, 2F, 2G, 2H and 2I.

The sensor is formed on a first, device wafer 210. The device wafer 210 in one embodiment may be an undoped silicon (100) wafer on which approximately 1.5 microns of thermal oxide 215 may be grown. A first layer of lithography may be used to define trenches 220 in the oxide 215. These may be created using reactive ion etch (RIE) techniques with photoresist as an etch mask. The trenches may act as anchor points for subsequently formed resonator devices and also may serve to expose the silicon substrate beneath the oxide.

Following the creation of the trenches, a low stress silicon nitride layer 225 may be deposited using low pressure chemical vapor deposition (PRCVD). The optimal thickness of these layers (oxide and nitride) may be calculated such that the effects of the detection method on resonant frequency are minimized as discussed below. The device thickness in one embodiment is approximately 220 nm, but other thicknesses may be used.

The nitride layer or film 225 may be patterned using photolithography and RIE to define the body of the resonators 230. The thermal oxide 215, referred to as a sacrificial oxide, may then be removed such as by using a wet hydrofluoric acid (HF) etch. This releases the resonators 230. Residual photoresist is then removed in one embodiment, such as by first chemically stripping the resist in a hot (75 C) bath of N-methylpyrolidone (Microposit 1165) for 20 minutes followed by a two minute oxygen plasma descum.

At least two different methods of encapsulating the device with a second wafer 250 may be used. In a first method, the second wafer may be a 500 micron thick borosilicate glass. The second wafer 250 may be first coated with 300 nm of amorphous silicon 255 on both sides using plasma enhanced chemical vapor deposition (PECVD) at 300 C. Prior to this, wafer 250 was cleaned in a 1:1:6 mixture of ammonium hydroxide, hydrogen peroxide, and deionized water to promote adhesion of the a-Si layer 255. This layer may be patterned at 260 with photolithography and RIE to define a channel. The remaining a-Si acts as a mask against an HF we etch which defined the 20 micron deep, 150 micron wide channels 265. The mask layer 255 may then be removed using RIE. Although smaller channels may be formed, the above dimensions accommodate arrays of NEMS sensors. Ports may be formed into the glass wafer by sand blasting or other means. The first and second wafers may then be anodically bonded as illustrated in FIG. 2G. The combined wafers provide a channel that is sufficiently sealed to enable creation of at least a partial vacuum for sensing vibrations of the sensor when an analyte is attached.

In a second method of encapsulation, channels may be defined on both the device wafer 210 (before device release), and the second wafer 250 through patterning of 14 μm thick SU8 2010 negative resist 270 resulting in a channel wall 275 on second wafer 250 and a channel wall 280 on wafer 210. The devices on device wafer 210 may then be released using hydrofluoric acid. Subsequently, the wafers may be aligned such that walls 275 and 280 are aligned to form the channel. The wafers are then bonded together by applying pressure (˜20 kPa) at 200 C for 30 minutes.

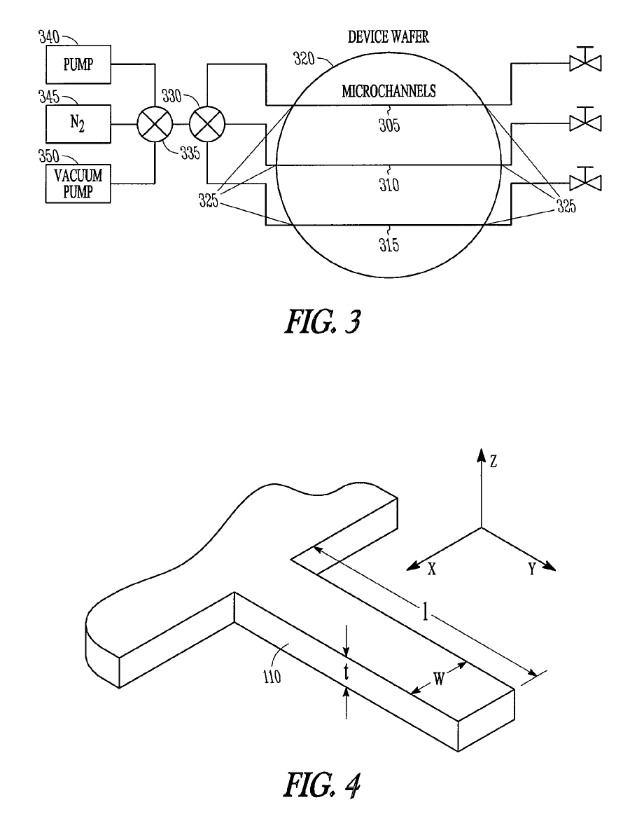

FIG. 3 is a schematic diagram illustrating plumbing for implementing various methods of using channels 305, 310 and 315 on a device wafer 320 with integrated resonators. Microfluidic fittings 325 may be made out of polyetheretherketone (PEEK) and may be attached to the channels using an epoxy resin in one embodiment. Other suitable plumbing connections and off-chip components to the channels may be made using components made of PEEK or other materials. A first selectable inlet/outlet valve 330 is coupled between the channels and a second selectable inlet/outlet valve 335. Second valve 335 is coupled to a pump 340, an N₂ source 345 and a vacuum pump 350. Single channel valves are coupled to the other end of each channel to allow flow or seal the end of a channel as desired. In further embodiments, many of these components, such as the valves may be formed and integrated onto the wafer with the devices.

In one embodiment, the channel access ports may be connected to the two multiport valves in series, allowing for multiplexed control of fluid transport. Sample delivery may be accomplished using pump 340, which may be a syringe pump in one embodiment. The sample may be selectively delivered by use of the multiple valves. When filling the channel with sample, a corresponding valve 325 may be opened to permit flow.

Following sample delivery, the channels may be purged and dried. In one embodiment, a high-purity dry nitrogen source 345 may be coupled to a channel via the valve network, again with the corresponding valve 325 open to allow flow. The source 345 may contain any gas that operates to purge and dry the channels after liquid sample delivery, and not interfere with binding of the desired amount of analyte to the resonator.

Following purging and drying, the channels may be pumped down by pump 350 to a pressure where viscous damping effects are negligible, thus encapsulating the devices within the microfluidic channels. This is referred to as a partial vacuum in one embodiment. In one embodiment, the pressure may be approximately less than 1 mTorr when measured at pump 350. Pump 350 in one embodiment may be a rotary-vane vacuum pump coupled to the multi-port valves 335 and 330. In one embodiment the vacuum should be sufficient such that the quality factor of the resonator does not significantly degrade from that in a pure vacuum.

In one embodiment three different resonator device lengths may be used, 7 μm, 10 μm and 12 μm. These lengths were selected for example purposes only, and should not be taken as limits. Each resonator in these example embodiments is about 3 μm wide, and exhibited resonant frequencies of 2.0, 3.1 and 6.3 MHz, respectively.

In the partial vacuum, the resonators may be actuated or caused to resonate thermally in one embodiment. In further embodiments, other means of actuation may be utilized, such as piezoelectric methods or still other methods. Nanometer scale motion of then sensors may be measured using an optical interferometric technique in one embodiment. In further embodiments, spectrum analyzer 160 may be used.

Since the NEMS resonators consist of a stack of thin films, device motion changes the thickness of a gap between the device and the substrate, causing modulation of the light reflected off the stack. Using an HeNe laser source 130 with a wavelength of 632.8 nm and a power output of 20 mW, a beam 140 may be directed from the source to the device wafer using mirrors and focusing optics. The light reflected off the sample may be measured using an AC coupled New Focus 1601 1 GHz bandwidth photodetector in one embodiment. These elements may also be placed on a vibration isolation table to reduce vibration noise.

In still further embodiments, the resonator may be formed of a waveguide, and a photodetector located opposite the cantilevered end of the waveguide to measure changes in light received as the cantilever oscillates. A mirror may also be located opposite the cantilevered end of the waveguide with photodetector optically coupled to the waveguide to receive reflected light.

In one embodiment, a second laser source, such as a diode providing 405 nm, 12 mW light, may be used to drive the devices. The output of this laser may be modulated using an electro optical modulator. Its beam may be focused on or near the device under test, acting as a modulated heat source, creating a localized stress field that causes resonance of the device when modulated at the natural frequency of the device.

In one embodiment, beam powers and film thicknesses may be controlled and selected in a manner to minimize absorption of incident laser light to avoid adding significant DC thermal input to the resonator. Further reduction of DC thermal input may be achieved by defocusing beam spots.

In one embodiment, the response of the device to the driving signal may be measured by reading the output of the photodetector using a network analyzer while simultaneously using the amplified signal from the analyzer's tracking generator to modulate the drive laser and thereby actuate the device. Frequency and quality factor values may be extracted by filtering the measured spectrum to a Lorentzian curve using a nonlinear Levenberg-Marquardt fitting algorithm.

In one embodiment, the physical dimensions of beam 110 may be selected to meet desired sensitivity requirements. FIG. 4 illustrates the geometry of a typical beam 110. In various embodiments, beam 110 has a high aspect ratio, that is the length l, is longer than the width w, of beam 110. By way of example, but not by way of limitation, a high aspect ratio beam is one having a ratio of length to width of approximately 3.75 or more. For example, typical dimensions for the length of beam 10 can be in the range of 0.5 to 1000 μm. Typical dimensions for the width of beam 10 can be in the range of 0.1 to 50 μm. A typical dimension for the thickness t, of beam 10 can be in the range of 0.05 to 4 μm. The aforementioned dimensions are not to be construed as limitations. A coordinate system is also illustrated in FIG. 4, with the z-axis aligned with t, the x-axis aligned with w, and the y-axis aligned with 1.

In the embodiment shown in FIG. 1, beam 110 vibrates in the directions of arrow 115, or substantially along the z-axis. Arrow 115 extends normal to the plane of beam 110, and thus, the vibratory mode is said to be out of plane. Other modes of vibration may also be sensed. For example, vibrations in plane may be monitored with suitable sense apparatus. Vibrations in more than one plane can also be monitored.

Support 120 is coupled to one end of beam 110. In the embodiment shown in FIG. 1, support 120 is illustrated as a rectangular housing. Support 120 can be a region of the substrate upon which cantilever beam 110 is fabricated, and is thus stable relative to the vibrations of cantilever beam 110. Support 120 can be fabricated in conjunction with the fabrication of beam 110. Consequently, support 120 may also be fabricated of the same material used in the fabrication of beam 110. In addition, support 120 may be fabricated in conjunction with other integrated electronic devices, components or circuitry. The other integrated electronic devices, components or circuitry may be related or unrelated to the operation of detector system 100. For example, support 120 may be fabricated on the same substrate as digital logic gates, amplifiers, processors, memory cells, or other semiconductor devices.

Cantilever beam 110 vibrates at frequencies determined by the geometry, the mass, the distribution of mass, and external forces acting on beam 110. A change in the mass of beam 110 is detectable as a change in one or more resonant frequencies of beam 110. In one embodiment this phenomena may be used for a particular beam sensitized for detecting E. coli cells. The number of cells coupled to the beam is proportional to the mass change of beam 110. A substantially linear relationship exists between mass and frequency differential. Deviations from linearity may be explained by such factors as nonuniform loading of beam 110 as well as nonuniform flexural rigidity of beam 110 resulting from variations in the distribution of the mass of beam 110.

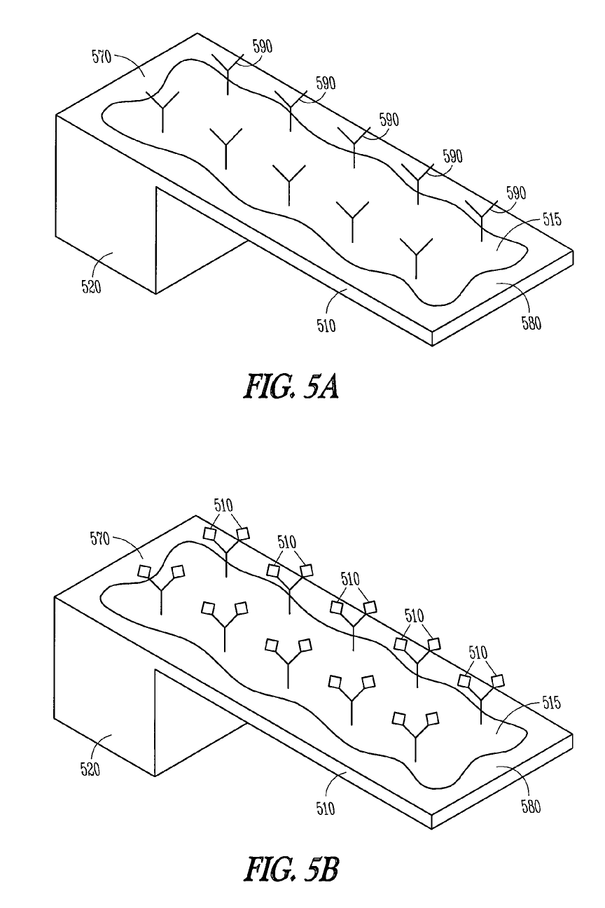

FIGS. 5A and 5B illustrate one embodiment of cantilever beam 510 having a binding partner 515 on a surface. It should be noted that the encapsulated resonator with integrated microfluidics may be used for many different purposes, and the use of a binding partner as described is just one of such uses. In the figures, support 520 is represented as a base structure and is rigidly attached to further structure not appearing in the figure. Beam 510 has a first end 570 rigidly attached to support 520 and a second end 580 that is cantilevered. In one embodiment, second end 580 is free to vibrate in an out of plane mode. Binding partner 515 is immobilized on beam 510. Binding partner 515 may be conformally distributed, or coated, on all surfaces of the structure illustrated in FIG. 5A. Binding partner 515 may also be localized to a particular portion of beam 510, such as, for example, a region near second end 580. Binding partner 515 can be distributed on an upper surface of beam 510. Binding partner 515 can be distributed on the exterior surfaces of beam 510. Binding partner 515 can be impregnated within the interior structure of beam 510. Binding partner 515 can be a surface coating on beam 510 and thus, selectively bind to predetermined molecules. In one embodiment, binding partner 515 includes molecules 590 that bind to complementary molecules on target cells in a “lock and key” fashion. In the embodiment of FIG. 5A, binding partner 515 includes a plurality of antibody molecules, herein represented as a plurality of “Y” shaped characters 590. FIG. 5B illustrates beam 510 having binding partner 515 at a time when complementary molecules 510 have bound with the antibody molecules 590 of binding partner 515. Binding partner 515 can bind to one or more target substances in a reversible or essentially irreversible fashion. Examples of essentially irreversible bonds may include those arising by van der Waal forces, ionic bonds, or by formation of covalent bonds. Preferably, binding does not occur by simple physical absorption of the target by beam 510 or binding partner 515 thereon.

In FIG. 5A, one embodiment of beam 510 is shown having an amount of binding partner 515 immobilized on the surface. Binding partner 515 is selected to bind to a desired target substance, or substances, wherein the bound target substance, or substances, is then detected as in system 100. For example, one protein (such as an antibody) may be used as a binding partner 515 on beam 510 for purposes of detecting a second protein (such as an antigen). By way of example only, and not by way of limitation, other pairs include using a receptor for detecting a ligand such as using a cellular receptor to detect a ligand that binds to such receptor, using a protein for detecting a peptide, using a protein for detecting a DNA, using a first DNA sequence to detect a second DNA sequence, using a metallic ion to detect a chelator, and using an antibody, or an antibody fragment, for detecting an antigen or analyte. It will be recognized that the aforementioned examples bind to each other in a “lock and key” fashion by ionic bonding, covalent bonding or a combination thereof. In some cases, the binding partner may bind specifically to a single target substance or subunit thereof. Consequently, either the “lock” can be immobilized on beam 510 for detecting the “key” or the “key” can be immobilized on beam 510 for detecting the “lock.” As an example, a peptide may be the binding partner on beam 510 for use in detecting a protein. The binding partner 515 immobilized on cantilever beam 510 can be DNA and thus, the present system is responsive to the substantial DNA complement. The bound, or “hybridized” DNA sequences can then be treated or “washed” under various conditions of stringency so that only DNA sequences that are highly complementary (e.g., that has high sequence identity) will be retained on beam 510.

The binding partner 515 can also bind to a plurality of substances, in which case, system 100 will indicate detection of any substance binding to cantilever beam 510. In addition, more than one binding partner 515 may be immobilized on a particular cantilever beam 510 to enable detection of multiple molecules. Multiple binding partners 515 may be immobilized in the same or different regions of cantilever beam 510.

The binding partner 515 can include an antibody for detection of an antigen, or binding partner 515 includes an antigen for detection of an antibody. Examples of antigens include proteins, oligopeptides, polypeptides, viruses and bacteria. For instance, antigens include OMPa, OMPb and OMPc, commonly referred to as outer membrane protein “a” “b” and “c.” In such cases involving antigens, the interaction includes one or more amino acid interactions wherein the amino acids are spatially arranged to form two complementary surfaces in three dimensions. Each surface includes one or more amino acid side chains or backbones.

The binding partner 515 can include an antibody for detection of a hapten, or binding partner 515 includes a hapten for detection of an antibody. Haptens tend to be much smaller than antigens and include such compounds as transition metal chelators, multi-ring phenols, lipids and phospholipids. In such cases involving haptens, the interaction includes an intermolecular reaction of a surface of the hapten with one or more amino acids of the antibody, wherein the amino acids of the antibody are spatially arranged to form a complementary surface to that of the hapten.

The interaction between amino acids, such as antibody-antigen or antibody-hapten, arises by van der Waal forces, Lennard-Jones forces, electrostatic forces or hydrogen bonding. Consequently, immobilized binding partner 515 interacts with the targeted substance in a manner beyond that of simple absorption of analyte into a matrix of some type. The interaction of binding partner 515 with the target substance is characterized by rapid bonding, preferably bonding that is not reversible under ambient conditions, thus reducing the time required for reliable detection using system 100.

Hybrid antibodies are also contemplated for either the target substance or binding partner 515. For example, a portion of a first antibody may be cleaved and a second antibody may be bonded to the remaining portion of the first antibody, thus forming a hybridized antibody. Such an antibody may subsequently bind with two forms of antigens or haptens. As yet another example, a third antibody may be bonded to the remaining portion of the first antibody, thus enabling subsequent bonding to additional antigens or haptens. The use of hybridized antibodies in system 100 yields a detector sensitive to multiple substances and may be desirable for certain applications where detection of two or more analytes is desired.

Binding partner 515 may be affixed, or immobilized, to the surface of beam 510 using any of a number of techniques, including absorption, covalent bonding with or without linker or spacer molecules or complexation.

Other methods for immobilizing binding partner 515 to beam 510 are also contemplated. For example, binding partner 515 can be covalently bonded to a surface of beam 510. Binding partner 515 can also be non-covalently bonded to a surface of beam 510. Binding partner 515 can be bonded by absorption to a surface of beam 510. In particular, amino chemistry, carboxyl chemistry, and carbohydrate chemistry techniques may be used to bond binding partner 515 to a surface of beam 510. A beam having a bound immobilized binding partner may be referred to as functionalized beam or resonator.

In further embodiments, binding sites are prefabricated on localized areas of beam 510. The binding sites provide an increased selectivity for a desired substance, allowing it's mass to be detected due to resonant frequency shifts of the resonators. In one embodiment, prefabricated catalyzing adsorption sites are incorporated into small resonators. The sites may be formed of precisely positioned gold anchors on the resonators. The resonators may be formed of silicon, such as polysilicon, or silicon nitride in various embodiments. The sites allow special control of chemical surface functionality for the detection of analytes of interest. In various embodiments, the sites reduce the amount of nonspecifically bound material, thus increasing sensitivity of mass measurements.

Arrays of oscillators or resonators may be fabricated using photolithographic processes, such as electron beam lithograph (EBL). The sites may be formed by evaporating gold. In addition, the method of fabricating may be easily adapted for wafer level vacuum packaging. In one embodiment, Thiolate molecules may be adsorbed from solution onto the gold anchors, creating a dense thiol monolayer with a tail end group pointing outwards from the surface of the gold anchor. This results in a thiolate self-assembled monolayer (SAM), creating a strong interaction between the functional group and the gold anchor.

In further embodiments, selective amounts of gold may be removed form the gold anchors to obtain desired frequency response characteristics. Further, precise tailoring of the length of the alkane chain and chemical properties of both head and tail groups provide excellent systems for further engineering of the chemical surface functionality following assembly of the SAM.

Other means of deriving, or analyzing, the frequency response of a cantilever beam 510 may be used in further embodiments. In one embodiment, movement of the cantilever beam 510 is detected based on a change in capacitance. For example, cantilever beam 510 serves as one electrode of a capacitor and a second electrode is held in a fixed position near the cantilever beam. Capacitance between the first and second electrode will vary as a function of the movement of cantilever beam 510. As another example, movement of cantilever beam 510 may be used to change the thickness, or amount, of dielectric material between beam 510 and a stationary electrode. Changes in dielectric thickness, or amount, are measurable as a frequency response. In one embodiment, piezoelectric or piezoresistive methods are used to detect the movement of cantilever beam 510. Piezoelectric detection involves generating an electric signal when the material is subjected to stress and piezoresistive detection involves sensing changes in resistance based on a stress in cantilever beam 510. Magnetic detection involves conductor movement relative to a magnetic field. Current in the conductor may be sensed. Cantilever beam 510 can serve as the moving conductor in a stationary magnetic field.

The output of the sensor can be digitized and communicated to a processor which is also represented at 160. The processor may use programming to discern the differential frequency, and thus the mass difference. The processor may further execute code to control the valves and pumps and sensing devices to perform a complete process of providing analyte to desired resonators in the channels, purging and drying the sensors, creating at least a partial vacuum about the sensors and performing optical or other sensing of the resonators to determine the presence and/or type of bound analyte.

Each of the aforementioned methods of detecting the frequency response may be used in an embodiment of the present system. For example, multiple optical sensors may be used for an array of a plurality of cantilever beams. Alternatively, a single optical sensor may be used to monitor an array of a plurality of cantilever beams or other types of resonators.

In one embodiment, an array of cantilever beams is fabricated wherein some beams are tailored to detect a first type of cell and a second set of beams are tailored to detect a second type of cell. For example, the aspect ratio of a cantilever beam may be selected to respond with greater sensitivity to a cell having a particular mass. Geometric dimensions, the method of fabrication, and the material selected for the cantilever beam are some of the parameters that may be tailored to achieve a desired sensitivity.

In addition, the environment in which beam 510 operates has an effect on the sensitivity of the present subject matter. In the viscous regime, for example, the atmospheric pressure operating on beam 10 will produce a dampening effect due to the viscosity of the air. Increased dampening effects will degrade the sensitivity of the detector. The quality factor Q of cantilever beam 10 is proportional to the inverse square root of the atmospheric pressure. In one embodiment, a beam operating in an environment of atmospheric pressure of 1 atm (approximately 760 mm Hg) and at room temperature (approximately 25 C), may have a quality factor Q of between 5 and 8. With a Q in this range, a particular beam 510 can detect approximately 44 bound cells of bacteria, such as E. coli bacteria. Sensitivity increases with increased quality factor Q. Increased sensitivity of the present subject matter can enable detection of both single E. coli bacteria and single monoatomic layers.

In the molecular regime, on the other hand, the quality factor is inversely proportional to the pressure. Therefore, when operated in a vacuum of 1 mTorr at room temperature, the quality factor Q is on the order of 104 for one embodiment. When operated in such a vacuum, the present subject matter can detect a mass in the range of 14.8×10⁻¹⁵ grams or less, and when operated in a standard atmosphere, can detect a mass 100 times larger. In addition, the mass distribution on the length of beam 510 will affect sensitivity.

The resolution of the frequency spectra is related to the width of the peak, and thus, the quality factor Q. Resolution can be 0.1 Hz when operating in a vacuum and 10 Hz in standard atmosphere.

1. A device comprising:
a pair of bonded wafers;
a microfluidic channel disposed within the bonded wafers; and
a nanoelectromechanical mass detector encapsulated within the microfluidic channel.

2. The device of claim 1 and further comprising multiple nanoelectromechanical mass detectors encapsulated within the microfluidic channel.

3. The device of claim 1 and further comprising multiple microfluidic channels disposed within the bonded wafers and multiple nanoelectromechanical mass detectors encapsulated within each microfluidic channel.

4. The device of claim 1 wherein the nanoelectromechanical mass detector comprises a resonator with a binding partner adapted to modify a resonant frequency of the resonator when a desired analyte binds to it.

5. The device of claim 4 wherein the resonator comprises a cantilevered beam.

6. The device of claim 4 wherein the resonator is a sub-attogram mass detector.

7. The device of claim 1 wherein the pair of bonded wafers comprise a device wafer on which the mass detector resides, and a channel wafer in which the channel is formed.

8. The device of claim 1 wherein the pair of bonded wafers comprise a device wafer on which the mass detector resides and a portion of the channel is formed, and a channel wafer in which another portion of the channel is formed, wherein the wafers are aligned such that the portions of the channel mate to form a single channel.

9. A device comprising:
a substrate;
a microfluidic channel supported by the substrate and adapted to operate at less than approximately 1 mTorr; and
a nanoelectromechanical mass detector encapsulated within the microfluidic channel.

10. The device of claim 9 and further comprising multiple nanoelectromechanical mass detectors encapsulated within the microfluidic channel.

11. The device of claim 9 and further comprising multiple microfluidic channels supported by the substrate and multiple nanoelectromechanical mass detectors encapsulated within each microfluidic channel.

12. The device of claim 9 wherein the nanoelectromechanical mass detector comprises a resonator with a binding partner adapted to modify a resonant frequency of the resonator when a desired analyte binds to it.

13. The device of claim 12 wherein the resonator comprises a cantilevered beam.

14. A method of detecting an analyte, the method comprising:
delivering a sample which may contain an analyte via a microfluidic channel to a nanoelectromechanical mass detector encapsulated within the microfluidic channel;
creating a pressure within the microfluidic channel that significantly reduces viscous damping effects on the mass detector;
actuating the nanoelectromechanical mass detector; and
measuring a response of the nanoelectromechanical mass detector.

15. The method of claim 14 and further comprising purging and drying the mass detector after exposure to the sample and prior to creating the pressure to reduce viscous damping.

16. The method of claim 15 wherein the purging and drying is performed with N₂.

17. The method of claim 14 wherein the mass detector is actuated by providing localized heating to induce vibration of the mass detector.

18. The method of claim 14 wherein the response is measured by a spectrum analyzer to determine a shift from a resonant frequency of the mass detector from before exposure to the sample.

19. The method of claim 14 wherein the mass detector has been functionalized with an immobilized binding partner.

20. A method of detecting analytes, the method comprising:
delivering a sample which may contain at least one analyte via at least one microfluidic channel of multiple fluidic channels to one or more nanoelectromechanical mass detectors in an array of mass detectors that is functionalized with an immobilized binding partner and encapsulated within the at least one microfluidic channel;
purging and drying the at least one mass detector following delivery of the sample;
creating a pressure within the at least one microfluidic channel that significantly reduces viscous damping effects on the at least one mass detector;
actuating the at least one nanoelectromechanical mass detector; and
measuring a response of the at least one nanoelectromechanical mass detector to determine the presence of an analyte.

21. The method of claim 20 wherein different mass detectors in the array of mass detectors may be functionalized to bind with different analytes.
